(19) United States
(12) Patent Application Publication
(10) Pub. No.: US 2018/0117206 A1
(43) Pub. Date: May 3, 2018

(54) ENCLOSURE FOR POROUS MEMBERS IMPREGNATED WITH VOLATILE SUBSTANCE

(71) Applicant: Eddie N. Vega, Yonkers, NY (US)

(72) Inventor: Eddie N. Vega, Yonkers, NY (US)

(21) Appl. No.: 15/795,024

(22) Filed: Oct. 26, 2017

Related U.S. Application Data
(60) Provisional application No. 62/414,672, filed on Oct. 29, 2016.

(51) Int. Cl. A61L 9/12; A01M 1/20

(52) U.S. Cl. CPC A61L 9/12; A61L 2209/15; A61L 2209/134; A01M 1/2055

(57) ABSTRACT

An enclosure for porous members impregnated with volatile substances, such as odor-destroying, air-perfuming, or insect-destroying substances, which may be opened to the extent desired through the use of a slider controlled by the user, to permit the controlled exposure of said porous member to the ambient.

This application claims priority from U.S. Provisional Application 62/414,672, filed Oct. 29, 2016.

### BACKGROUND OF THE INVENTION

This invention relates to improvements in enclosures for porous members impregnated with volatile substances, and in particular, odor-destroying, air-perfuming, or insect-destroying substances, wherein the volatilization of such substances can be retarded or controlled.

Certain substances, such as odor-destroying, air-perfuming, or insect-destroying substances, are generally quite volatile. Porous members impregnated with such substances have heretofore been enclosed in impervious envelopes or pouches from which such members may be partially withdrawn to avoid excessive volatilization. An example of this technology is disclosed in U.S. Pat. No. 3,065,915 to Samann. Although the extent to which the impregnated members should be withdrawn from the pouches is typically indicated on packaging, frequently purchasers of these articles do not realize the rate of volatilization. As a result, even when controlled by withdrawing the impregnated member only partially from the pouch, volatilization can be too rapid, producing an excessively heavy and consequently unpleasant odor, and reducing the life of the impregnated member.

### OBJECTS AND SUMMARY OF THE INVENTION

It is therefore an object of the invention to provide an enclosure for porous, impregnated members of this kind, which may be opened to the extent desired through the use of a slider controlled by the user, to permit the controlled exposure of said porous member to the ambient. The slider can be controlled by the user so that the enclosure may either be fully closed, partially opened, or fully opened, so that the user of the device may control the extent the member should be exposed to the atmosphere, thus controlling the volatilization of the porous impregnated member.

The present invention finds particular applicability for use in controlling the volatilization of odor-destroying, air-perfuming, or insect-destroying substances.

In accordance with a first aspect of the present invention, an enclosure for a substantially planar member impregnated with a volatile substance comprises a case having two, substantially planar sides, the first side of which is provided with at least one opening. The case is adapted to receive the member between said first and second sides. A substantially planar sliding panel is disposed between and adapted to slide between the first and second sides of the case, and the sliding panel is positioned such that said volatile member is adapted to be between the second side of the case and the sliding panel. The sliding panel has at least one opening.

The openings in the first side of the case and the sliding panel are configured relative to each other such that the surface of the member adjacent to the sliding panel member is substantially isolated from the ambient when the sliding panel is in a first position relative to the case, and at least a portion of the surface of the member is substantially exposed to the ambient when the sliding panel is in a second position relative to said case. A projection is connected to and provides access to the sliding panel by the user of the enclosure, to thereby allow the user to selectively expose or isolate the surface of the member to or from the ambient.

In the preferred embodiment, the second side of said case is substantially solid, and the first and second sides of said case and the sliding panel are preferably made of a material that is substantially impermeable to the volatile substance. The first and second sides of the case can be connected by a hinge. The first side of the case can include a slot through which the projection connected to the sliding panel projects, so that the user can selectively expose or isolate the surface of the member to or from the ambient by sliding the projection within the slot.

The first side of the case and the sliding panel can be provided with a plurality of openings in substantially the same configuration, and in accordance with a preferred embodiment, the plurality of openings are substantially linear, parallel slots oriented substantially at a 90-degree angle with respect to the slot in the first side of said case.

The case can be shaped to accommodate a specific shape of said member, and may be adapted to enclose a member having an odor-destroying, air-perfuming, or insect-destroying volatile substance.

In accordance with a second aspect of the present invention, an enclosure for a member impregnated with a volatile substance comprises a case having two, substantially planar sides, and adapted to receive the member. A substantially planar sliding panel is disposed within the case, and means are provided for substantially isolating the member from the ambient when the sliding panel is in a first position relative to the case, and for exposing at least a portion of a surface of the member to the ambient when the sliding panel is in a second position relative to the case. Means are also provided for allowing the user of the enclosure to selectively change the position of the panel relative to the case.

### BRIEF DESCRIPTION OF THE DRAWINGS

These and other objects, aspects and features of the invention are described below with reference to the following drawing figures, which illustrate a preferred embodiment, and which are intended to illustrate, but not limit, the present invention:

FIG. 1 is a plan view of an example of an outer case used in connection with the present invention;

FIG. 2 is a plan view of the slider which is contained within the outer case of FIG. 1;

FIG. 3 is a plan view of an example of a porous, impregnated member, in this case an air freshener;

FIG. 4A is a plan view of the assembled enclosure in which the slider and porous member are disposed within the outer case, and the case is in the fully closed position;

FIG. 4B is a vertical cross-sectional view of the assembled enclosure of FIG. 4A;

FIG. 5A is a plan view of the assembled enclosure in which the slider and porous member are disposed within the outer case, and the case is in the fully opened position; and

FIG. 5B is a vertical cross-sectional view of the assembled enclosure of FIG. 5A.

### DETAILED DESCRIPTION OF THE PREFERRED EMBODIMENTS

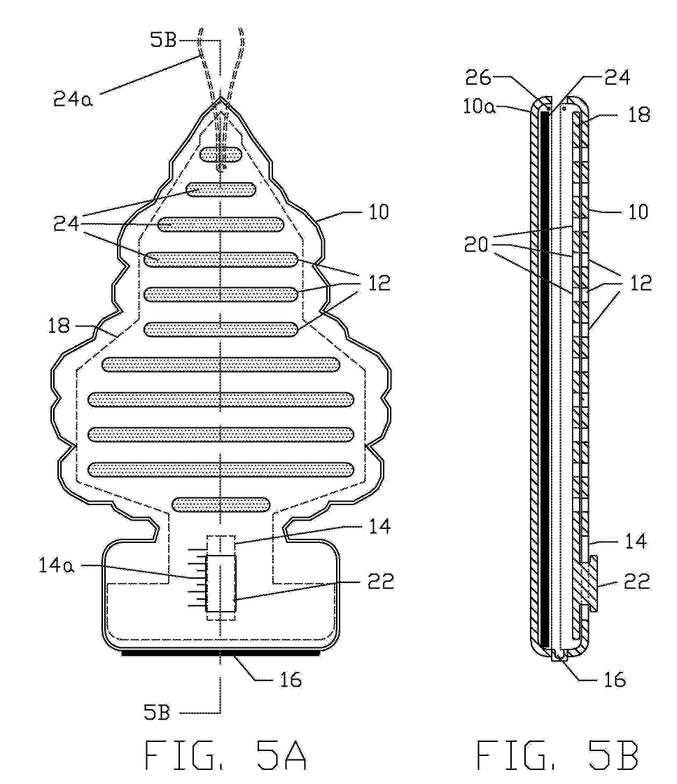

FIG. 1 illustrates a preferred embodiment of the outer case 10 of the invention, the front of which is provided with a plurality of horizontally disposed openings or slats 12 and a vertically disposed opening 14. Although shown as being horizontally and vertically disposed, respectively, the openings 12 and 14 are more generally disposed at 90-degree angles. The rear side 10a of case 10 (FIGS. 4A, 5A) is solid, not provided with any openings, and is connected to the front by means of and a hinge 16. The case 10 may be of any suitable material that is impervious to a particular volatile substance. In the example shown in the figures, the case 10 is specifically shaped to accommodate an air freshener, specifically, an air freshener sold under the trade name LITTLE TREES®. It will be appreciated, however, that the outer case 10 can be shaped to accommodate any particular porous member, or maybe shaped generically, so that a variety of differently shaped porous members may be used with the present invention.

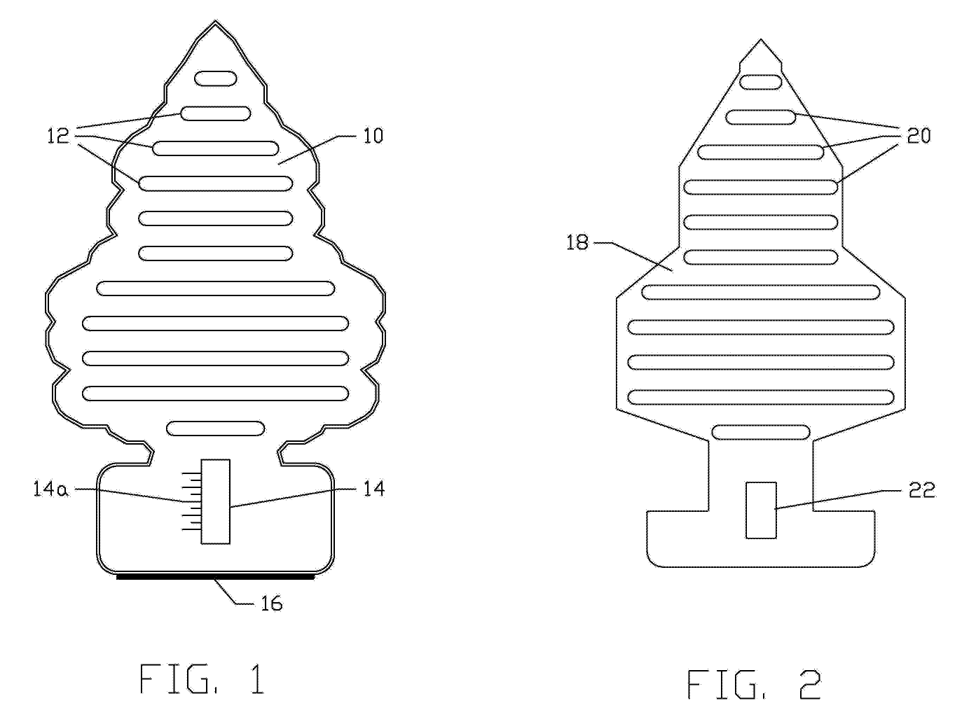

FIG. 2 illustrates the slider 18, which should be made of any suitable impervious material and typically will be made of the same material as the outer case 10. The slider 18 is provided with a plurality of horizontally disposed slats 20 that are configured to conform with the arrangement of slats 12 in the outer case 10. The slider 18 is also provided with a projection 22 which is adapted to protrude through the vertical opening 14 of the outer case 10.

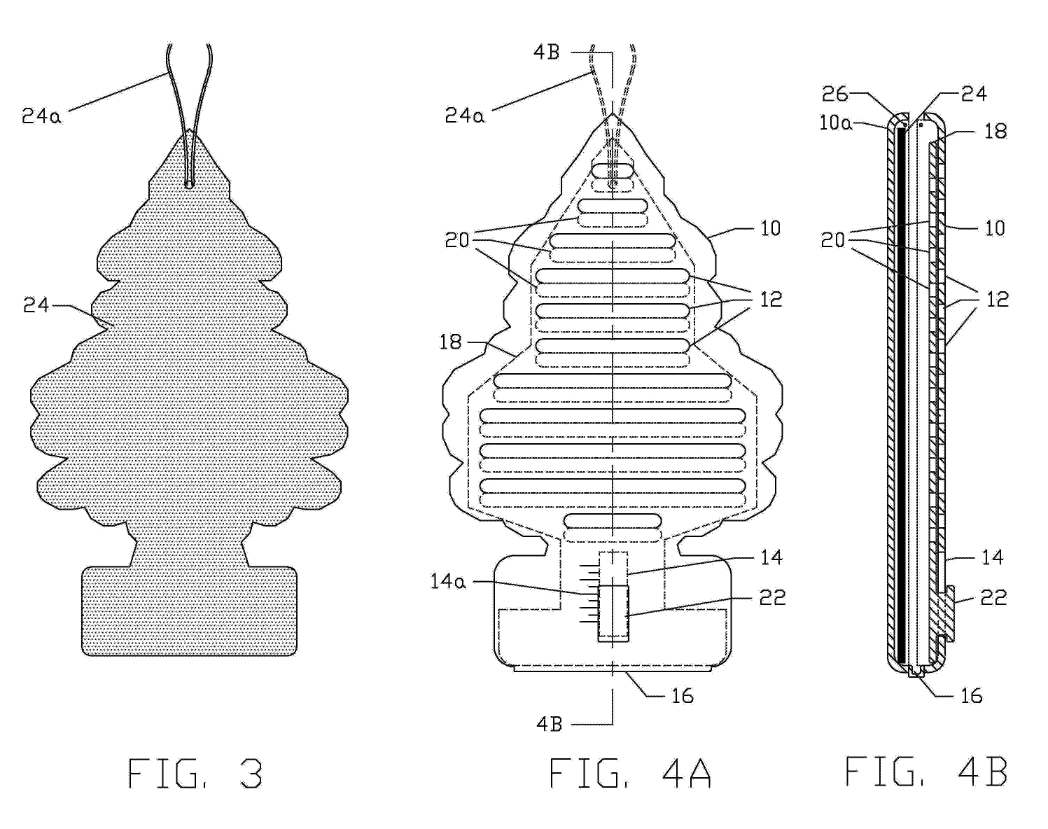

FIG. 3 illustrates an example of a porous, impregnated member 24, in this case a LITTLE TREES® air freshener. More generally, however, member 24 may be made of absorbent material such as employed in blotting or filter paper, and contains or is impregnated with a volatile substance, such as an odor-destroying, air-perfuming, or insect-destroying substance. It may be shaped differently from the example shown in FIG. 3, in which case the outer case 10 may be shaped to accommodate the member 24, or as previously mentioned, the outer case may be shaped generically, to accommodate a number of different configurations of member 24. The member 24 may be provided with a string 24a to allow it to be suspended, for example, from a rearview mirror in an automobile.

The structure and operation of the assembled enclosure will be described with reference to FIGS. 4A, 4B, 5A and 5B. The impregnated member 24 is disposed adjacent the rear side 10a of the case 10, which is connected to the front side of the case 10 by hinge 16. A snap, clip or other suitable closure 26 is provided at the top of the case 10 to substantially seal the case from the ambient. When the enclosure is in the closed position, as shown in FIG. 4B, and the slider 18 is in the position relative to the case 10, the enclosure is substantially sealed so that none or very little of the volatile material in the member 24 will escape from the enclosure. Specifically, in the position shown in FIGS. 4A and 4B, the slats 12 in the outer case 10 and the slats 20 in the slider 18 are not aligned, thus isolating the member 24 from the ambient. With reference to FIGS. 5A and 5B, the user may slide the projection 22, attached to the slider 18, within the opening 14 of the outer case 10, to thereby align the slats 12 and 20 to thereby expose the surface of the member 24 to the ambient. As shown in FIGS. 5A and 5B, the slats 12 in 20 are completely aligned with each other to thereby provide the maximum exposure of the member to the ambient. It will be appreciated, however, that the slider 18 may be adjusted by the user so that different gradations of exposure can be achieved, and the outer case 10 may be provided with visible indicia 14a, in order to guide the user to the extent to which the member should be exposed. Although the sliding action described above is linear, the term “sliding” as used herein is intended to cover rotational sliding, in the event that the slider is rotated within the case, by means of a dial or the like, to vary the exposure of the member.

Thus, the enclosure may initially be opened only slightly so that only a small portion of the member 24 is exposed to the ambient and the volatilization of the substances in member 24 will take place slowly. Over time, as the substances in the member are depleted, the user may increase the opening to thereby provide a substantially continuous, desired level of volatilization through the life of member 24. When the strength of the volatile material has greatly diminished, the member 24 can be reversed or entirely removed from the enclosure, and then be replaced with a new member.

The structure of the enclosure previously described may be modified by providing openings and a slide on both sides of outer case 10 to thereby increase the available level of volatilization. Either or both sides 10 or 10a may be provided with printed matter, on either the inside or outside surfaces of the outer case, or rendered at least partly opaque, or may be clear, or provided with a combination of the foregoing. In the event that printing is provided on the inner face or the case, and the volatile substance in the member has a detrimental effect on the printing, the case may be provided with a double front wall, the outer wall of which would be transparent, and the printed matter would be placed between the double front walls.

Thus, the present invention represents a significant improvement in the ability to control the exposure of the member 24. It results in a more even, desirable level of volatilization and may extend the life of the member. Further, the present invention makes it possible to impregnate member 24 with a relatively large quantity of volatile material so that the article can remain effective for deodorizing and perfuming an auto, room or other enclosure for a much longer period of time than would be the case if the member were impregnated with a smaller quantity of the volatile substance. By leaving the extent of exposure of the member from the enclosure entirely to the judgment of the user, he/she will control the strength of the odor release and remove the possibility of producing an unpleasantly strong odor.

It will be understood that various changes in the details, materials and arrangements of parts which have been herein described and illustrated in order to explain the nature of the invention may be made by those skilled in the art within the principle and scope of the invention as expressed in the following claims.

What I claim is:

1. An enclosure for a substantially planar member impregnated with a volatile substance, comprising:
a case having two substantially planar sides, the first side of which is provided with at least one opening, said case adapted to receive said member between said first and second sides;
a substantially planar sliding panel disposed between and adapted to slide between said first and second sides of said case, said sliding panel positioned such that said volatile member is adapted to be between said second side of said case and said sliding panel, said sliding panel having at least one opening;
the at least one opening in said first side of said case and the at least one opening in said sliding panel being configured relative to each other such that the surface of said member adjacent to said sliding panel is substantially isolated from the ambient when the sliding panel is in a first position relative to said case, and at least a portion of said surface of said member is substantially exposed to the ambient when said sliding panel is in a second position relative to said case; and
a projection connected to and providing access to said sliding panel by a user of the enclosure, to thereby allow the user to selectively expose or isolate said surface of the member to or from the ambient.

2. The enclosure of claim 1 wherein the second side of said case is substantially solid, and the first and second sides of said case and said sliding panel are made of a material that is substantially impermeable to said volatile substance.

3. The enclosure of claim 2 wherein said first and second sides of said case are connected by a hinge.

4. The enclosure of claim 3 wherein said first side of said case includes a slot through which said projection connected to said sliding panel projects, and wherein the user can selectively expose or isolate the surface of the member to or from the ambient by sliding the projection within said slot.

5. The enclosure of claim 4 wherein said first side of said case and said sliding panel are provided with a plurality of openings in substantially the same configuration.

6. The enclosure of claim 5 wherein said plurality of openings are substantially linear, parallel slats oriented substantially at a 90-degree angle with respect to said slot in said first side of said case.

7. The enclosure of claim 6 wherein said case is shaped to accommodate a specific shape of said member.

8. The enclosure of claim 5 wherein said case is adapted to enclose a member having an odor-destroying, air-perfuming, or insect-destroying volatile substance.

9. An enclosure for a member impregnated with a volatile substance, comprising:
a case having two, substantially planar sides, said case adapted to receive said member;
a substantially planar sliding panel disposed within said case;
means for substantially isolating said member from the ambient when the sliding panel is in a first position relative to said case, and for exposing at least a portion of a surface of said member to the ambient when said sliding panel is in a second position relative to said case; and
means for allowing the user of the enclosure to selectively change the position of the panel relative to the case.

10. The enclosure of claim 9 wherein the first and second sides of said case and said sliding panel are made of a material that is substantially impermeable to said volatile substance.

11. The enclosure of claim 10 wherein said first and second sides of said case are connected by a hinge.

12. The enclosure of claim 10 wherein said case is shaped to accommodate a specific shape of said member.

13. The enclosure of claim 10 wherein said case is adapted to enclose a member having an odor-destroying, air-perfuming, or insect-destroying volatile substance.
